(19) United States
(12) Patent Application Publication
(10) Pub. No.: US 2025/0325365 A1
(43) Pub. Date: Oct. 23, 2025

(54) VALVE REPLACEMENT DEVICE

(71) Applicant: BOSTON SCIENTIFIC SCIMED, INC., MAPLE GROVE, MN (US)

(72) Inventors: David Ryan, Claremorrs (IE); Tim O'Connor, Galway (IE); Donal Kyne, St Louis Park, MN (US); Daire Grant, Galway (IE); Scott Cook, Athenry (IE); Shane Conway, Longford (IE)

(73) Assignee: BOSTON SCIENTIFIC SCIMED, INC., MAPLE GROVE, MN (US)

(21) Appl. No.: 19/185,857

(22) Filed: Apr. 22, 2025

Related U.S. Application Data
(60) Provisional application No. 63/637,480, filed on Apr. 23, 2024.

(51) Int. Cl. A61F 2/24

(52) U.S. Cl. CPC A61F 2/2418; A61F 2220/0008; A61F 2220/0025

(57) ABSTRACT

Example valve replacement devices and methods of using valve replacement devices are disclosed. An example device includes a framework including a plurality of strut members forming a proximal anchor region. The proximal anchor region includes a plurality of distal crowns and a first connector crown positioned along a distal end of the proximal anchor region. The device further includes a first commissure assembly coupled to the distal end, wherein the first commissure assembly includes a first commissure post, a first connector strut and a second connector strut. The device further includes one or more leaflets mounted at least partly within the framework. Further, the first and second connector struts each include a first end attached to the first commissure post and a second end attached to the first connector crown at a first pivot point. Further, the pivot point is positioned radially outward of the plurality of proximal crowns.

### CROSS-REFERENCE TO RELATED APPLICATION

This application is a continuation of U.S. Patent Application Ser. No. 63/637,480, filed Apr. 23, 2024, entitled “VALVE REPLACEMENT DEVICE”, which is incorporated by reference herein in its entirety.

### TECHNICAL FIELD

The present invention is directed to devices for valve replacement and methods of using valve replacement devices, especially of the aortic valve.

### BACKGROUND

Conventional approaches for cardiac valve replacement may require the cutting of a relatively large opening in the patient's sternum (“sternotomy”) or thoracic cavity (“thoracotomy”) in order to allow a surgeon to access a patient's heart. Additionally, these approaches require arrest of the patient's heart and a cardiopulmonary bypass (i.e., use of a heart-lung bypass machine to oxygenate and circulate the patient's blood). Efforts have been made to develop less invasive cardiac valve replacement procedures, by delivering and implanting a cardiac replacement valve via a catheter inserted through a smaller skin incision via either a transvascular approach—delivering the new valve through the femoral artery, or by transapical route, where the replacement valve is delivered between ribs and directly through the wall of the heart to the implantation site.

Some replacement valves may be made from a shape memory material, such as Nitinol, and may be self-expanding. The replacement valve (e.g., a supra-annular replacement valve) may include multiple regions, whereby a distal region may be configured to anchor the valve and the proximal region houses the valve element. For a supra-annular replacement valve, the primary function of the distal region of the valve may be to anchor the implant and to create a seal against the native annulus/anatomy, while the function of the proximal end of the frame may be to support the valve leaflet function. The valve element may include three leaflets, which may be made of biological or artificial material.

When implanted during a transcatheter aortic valve replacement (TAVR) procedure, a supra-annular replacement valve may include an “effective orifice area,” defined as a measure of the cross-sectional area of the valve when in an open configuration. In a TAVR procedure, a preferred scenario may be to maximize the effective orifice area value to preserve optimal hemodynamic performance. Supra-annular replacement valves, with the leaflet coaptation point above the native annulus, may be inherently better at preserving the effective orifice area as the implanted leaflet shape is less affected by, for example, an elliptical native annulus at the point the lower valve frame is anchored against. Accordingly, replacement valve frame designs that include features which allow the proximal valve region to maximize an effective orifice area while the distal anchor region conforms and anchors to the shape of a native annulus are disclosed. The design of the distal and proximal regions may be optimized to enhance their respective functions.

### SUMMARY

This disclosure provides design, material, manufacturing method, and use alternatives for medical devices. An example valve replacement device for transcatheter implantation includes an expandable framework including a plurality of strut members spaced around a central longitudinal axis of the expandable framework and forming a proximal anchor region. Further, the proximal anchor region includes a plurality of distal crowns and a first connector crown positioned along a distal end of the proximal anchor region. The valve replacement device further includes a first commissure assembly coupled to the distal end of the proximal anchor region, wherein the first commissure assembly includes a first commissure post, a first connector strut and a second connector strut. The valve replacement device further includes one or more valve leaflets mounted at least partly within the expandable framework. Further, the first connector strut includes a first end attached to the first commissure post and a second end attached to the first connector crown at a first pivot point. Further, the second connector strut includes a first end attached to the first commissure post and a second end attached to the first connector crown at the first pivot point. Further, the pivot point is positioned radially outward of the plurality of proximal crowns.

Alternatively or additionally to any of the embodiments above, wherein a plurality of struts forming the proximal anchor region are configured to shift between a collapsed configuration and an expanded configuration.

Alternatively or additionally to any of the embodiments above, further comprising a second commissure assembly coupled to the distal end of the proximal anchor region, wherein the second commissure assembly includes a second commissure post, a second connector strut and a third connector strut.

Alternatively or additionally to any of the embodiments above, further comprising a third commissure assembly coupled to the distal end of the proximal anchor region, wherein the third commissure assembly includes a third commissure post, a fourth connector strut and a fifth connector strut.

Alternatively or additionally to any of the embodiments above, wherein the one or more valve leaflets include a first leaflet, a second leaflet and a third leaflet.

Alternatively or additionally to any of the embodiments above, wherein the first leaflet is coupled to the first commissure post and the second commissure post, wherein the second leaflet is coupled to the first commissure post and the third commissure post, and wherein the third leaflet is coupled to the second commissure post and the third commissure post.

Alternatively or additionally to any of the embodiments above, wherein the first leaflet, the second leaflet and the third leaflet are configured to shift from a sealed configuration to an open configuration.

Alternatively or additionally to any of the embodiments above, wherein the first leaflet, the second leaflet and the third leaflet define a maximum effective orifice area when in the open configuration.

Alternatively or additionally to any of the embodiments above, wherein the first commissure assembly, the second commissure assembly, and the third commissure assembly are configured to shift between a collapsed configuration and an expanded configuration.

Alternatively or additionally to any of the embodiments above, wherein the first commissure assembly, the second commissure assembly, and the third commissure assembly are configured to maintain the expanded configuration when the plurality of struts forming the proximal anchor region are in a partially collapsed configuration.

Alternatively or additionally to any of the embodiments above, wherein the first commissure assembly, the second commissure assembly, and the third commissure assembly are configured to maintain the maximum effective orifice when the plurality of struts forming the proximal anchor region are in a partially collapsed configuration.

Alternatively or additionally to any of the embodiments above, wherein the first commissure assembly, the second commissure assembly, and the third commissure assembly are configured to expand radially away from the central longitudinal axis when the struts forming the proximal anchor region are in the partially expanded configuration.

Alternatively or additionally to any of the embodiments above, wherein the first commissure assembly, the second commissure assembly, and the third commissure assembly are configured to extend away from the central longitudinal axis at an angle from 0 to 5 degrees.

Alternatively or additionally to any of the embodiments above, wherein the first commissure assembly pivots about the first pivot point to extend away from the central longitudinal axis at an angle from 0 to 5 degrees.

Another example valve replacement device for transcatheter implantation includes an expandable framework including a plurality of strut members spaced around a central longitudinal axis of the expandable framework and forming a proximal anchor region, wherein the proximal anchor region includes a plurality of distal crowns and a first connector crown positioned along a distal end of the proximal anchor region, and wherein the first connector crown includes a curved region which extends radially away from the central longitudinal axis. The valve replacement device further includes a first commissure assembly coupled to the distal end of the proximal anchor region, wherein the first commissure assembly includes a first commissure post, a first connector strut and a second connector strut. The valve replacement device further includes one or more valve leaflets mounted at least partly within the expandable framework. Further, the first connector strut includes a first end attached to the first commissure post and a second end attached to the first connector crown at a first pivot point. Further, the second connector strut includes a first end attached to the first commissure post and a second end attached to the first connector crown at the first pivot point. Further, the pivot point is positioned radially outward of the plurality of proximal crowns.

Alternatively or additionally to any of the embodiments above, further comprising a second commissure assembly and a third commissure assembly coupled to the distal end of the proximal anchor region, wherein the second commissure assembly includes a second commissure post, a second connector strut and a third connector strut, wherein the third commissure assembly includes a third commissure post, a fourth connector strut and a fifth connector strut.

Alternatively or additionally to any of the embodiments above, wherein the one or more valve leaflets include a first leaflet, a second leaflet and a third leaflet, and wherein the first leaflet is coupled to the first commissure post and the second commissure post, wherein the second leaflet is coupled to the first commissure post and the third commissure post, and wherein the third leaflet is coupled to the second commissure post and the third commissure post.

Alternatively or additionally to any of the embodiments above, wherein the first leaflet, the second leaflet and the third leaflet are configured to shift from a sealed configuration to an open configuration, and wherein the first leaflet, the second leaflet and the third leaflet define a maximum effective orifice area when in the open configuration.

Alternatively or additionally to any of the embodiments above, wherein the first commissure assembly, the second commissure assembly, and the third commissure assembly are configured to maintain the maximum effective orifice when the plurality of struts forming the proximal anchor region are in a partially collapsed configuration.

Another example valve replacement device for transcatheter implantation includes an expandable framework including a plurality of strut members spaced around a central longitudinal axis of the expandable framework and forming a proximal anchor region, wherein the proximal anchor region includes a plurality of distal crowns and a first connector crown positioned along a distal end of the proximal anchor region, and wherein the first connector crown includes a curved region which extends radially away from the central longitudinal axis. The valve replacement device further includes a first commissure assembly coupled to the distal end of the proximal anchor region, wherein the first commissure assembly includes a first commissure post, a first connector strut and a second connector strut. The valve replacement device further includes one or more valve leaflets mounted at least partly within the expandable framework. Further, the first connector strut includes a first end attached to the first commissure post and a second end attached to the first connector crown at a first pivot point. Further, the second connector strut includes a first end attached to the first commissure post and a second end attached to the first connector crown at the first pivot point. Further, the pivot point is positioned radially outward of the plurality of proximal crowns. Further, the first commissure assembly is configured to pivot about the pivot point to extend radially away from the central longitudinal axis.

The above summary of some embodiments is not intended to describe each disclosed embodiment or every implementation of the present disclosure. The Figures and Detailed Description, which follow, more particularly exemplify these embodiments.

### BRIEF DESCRIPTION OF THE DRAWINGS

A better understanding of the features and advantages of the methods and apparatuses described herein will be obtained by reference to the following detailed description that sets forth illustrative examples, and the accompanying drawings of which:

FIG. 1 illustrates an example embodiment of a valve replacement device;

FIG. 2 is a perspective view of an example valve replacement device in a first configuration;

FIG. 3 is a perspective view of an example valve replacement device in a second configuration;

FIG. 4 is a perspective view of the expandable frame of the valve replacement device shown in FIG. 1;

FIG. 5 illustrates a portion of the expandable frame shown in FIG. 4;

FIG. 6 is a top view of the frame of the valve replacement device shown in FIG. 1;

FIG. 7 illustrates another example valve replacement device;

FIG. 8 is a perspective view of the frame of the valve replacement device shown in FIG. 7.

### DETAILED DESCRIPTION

The following description should be read with reference to the drawings, which are not necessarily to scale, wherein like reference numerals indicate like elements throughout the several views. The detailed description and drawings are intended to illustrate but not limit the claimed disclosure. Those skilled in the art will recognize that the various elements described and/or shown may be arranged in various combinations and configurations without departing from the scope of the disclosure. The detailed description and drawings illustrate example embodiments of the claimed disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

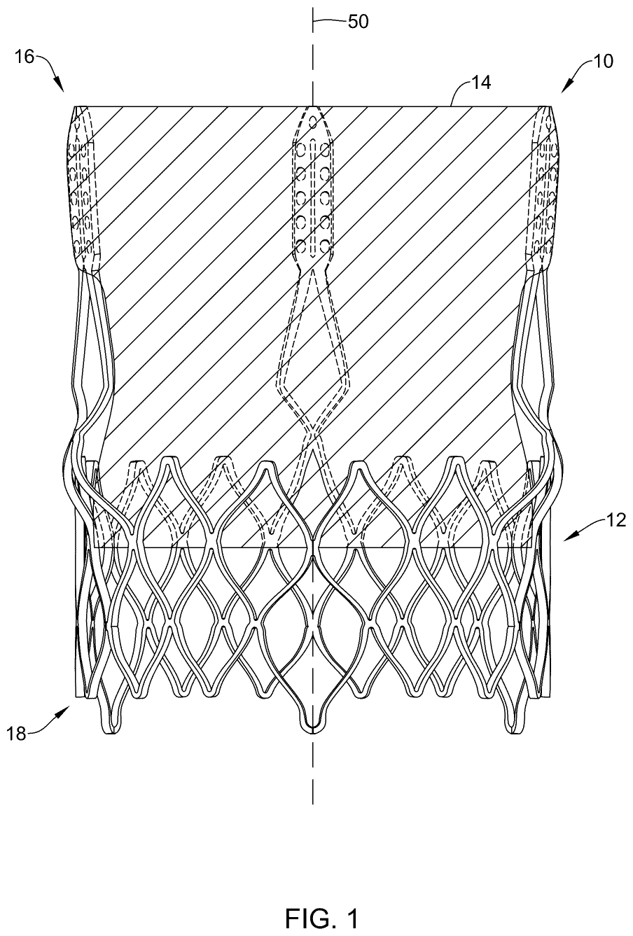

FIG. 1 illustrates an example valve replacement device 10 including an expandable frame 12 (e.g., a stent frame) and a valve component 14. The valve replacement device 10 may be configured to be inserted by a transfemoral approach, but the device may also be inserted generally by another transvascular approach or by a transapical approach. The valve replacement device 10 may be collapsed within a delivery catheter for tracking and deploying the valve replacement device 10 within the heart and/or the aorta. The delivery catheter may be advanced over a guidewire to a valve treatment site within the patient's heart. The delivery catheter may include a distal sheath component for constraining the valve replacement device 10 in a compressed state for delivery, the sheath arrangement being operable to un-sheath the valve replacement device 10 to allow the expandable frame 12 to expand to its functional state and nest within the native annulus of the patient.

FIG. 1 further illustrates that the valve replacement device 10 may extend circumferentially around a longitudinal axis 50 and include a first end 16 and a second end 18. In some examples, the first end 16 may be intended to be positioned in an artery, while the second end 18 may be intended to be positioned in or towards the ventricle of the heart of a patient. When the valve replacement device 10 is in place, blood may flow from the second end 18 toward the first end 16. Accordingly, the region of the valve 10 adjacent the second end 18 may be referred to as the “inflow section” and the region of the valve 10 adjacent the first end 16 may be referred to as the “outflow section.”

FIG. 1 further illustrates that the valve replacement device 10 may include a proximal region configured to anchor the valve replacement device 10 and a distal region configured to house a valve element 14. The terms “distal” and “proximal” are used herein to designate the parts of the valve replacement device 10 or of its components lying further away or closer to the heart, respectively. The distal end may also be referred to as the aortic end and the proximal end as the ventricular end. For a supra-annular valve replacement device 10, the primary function of the proximal region of the valve replacement device 10 may be to anchor the valve replacement device 10 and to create a seal against the native annulus/anatomy, while the function of the distal end of the valve replacement device 10 may be to support the valve leaflet function.

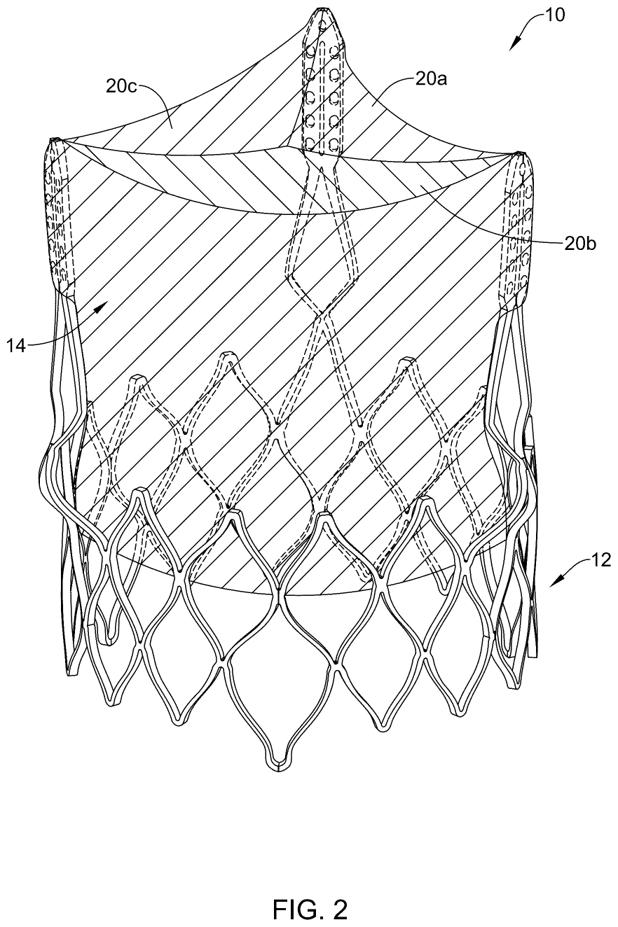

FIG. 2 illustrates that the valve element 14 may include three valve leaflets 20a, 20b, 20c, which may be made of biological or artificial material. For example, the three valve leaflets 20a, 20b, 20c may be formed from porcine pericardium tissue. Other similar materials are contemplated to form the three valve leaflets 20a, 20b, 20c. It can be appreciated that in some examples, the valve element 14 may include fewer than three valve leaflets while in other examples the valve element 14 may include more than three valve leaflets. For example, the valve element 14 may include 1, 2, 3, 4, 5, 6, or more leaflets.

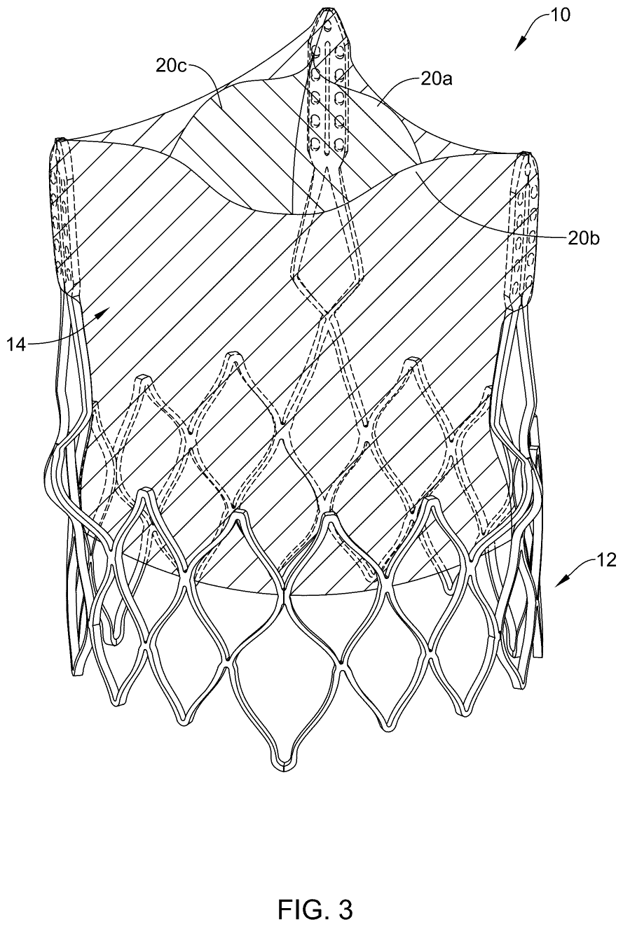

Further, the three valve leaflets 20a, 20b, 20c may be configuration to shift between a tight-sealed, closed configuration (shown in FIG. 2) and an open configuration (shown in FIG. 3). It can be appreciated that in the closed configuration, a free edge of each of the valve leaflets 20a, 20b, 20c may contact and seal against a portion of a free edge of one or more of the adjacent valve leaflets 20a, 20b, 20c. For example, in the closed configuration, a free edge of each of the valve leaflet 20a may contact and seal against a portion of the free edge of one or more of each adjacent valve leaflets 20b, 20c. Further, in the closed configuration, a free edge of each of the valve leaflet 20b may contact and seal against a portion of the free edge of one or more of each adjacent valve leaflets 20a, 20c. Further, in the closed configuration, a free edge of each of the valve leaflet 20c may contact and seal against a portion of the free edge of one or more of each adjacent valve leaflets 20a, 20b.

FIG. 3 illustrates a configuration in which the valve leaflets 20a, 20b, 20c of the valve element 14 are in an open configuration whereby blood may pass from the left ventricle of a patient's heart, through the valve element 14 and into the aorta of the patient's heart. It can be appreciated from FIG. 3 that in the open configuration the free edge of each of the adjacent valve leaflets 20a, 20b, 20c which seals against adjacent free edges of the valve leaflets 20a, 20b, 20c (as described herein) have been spaced away from one another to create the opening in which blood may flow from the patient's left ventricle and into the patient's aorta.

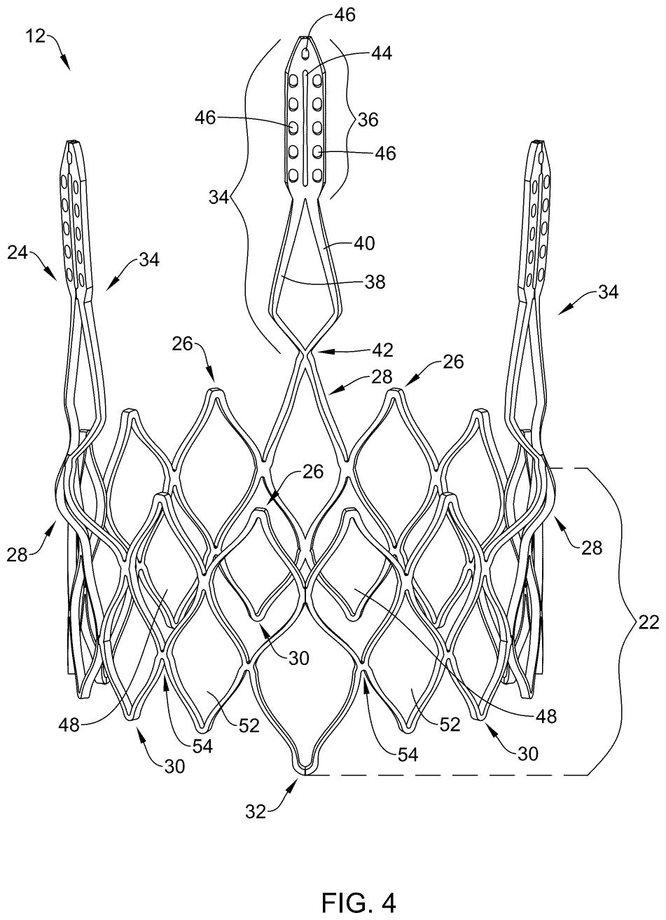

FIG. 4 illustrates the expandable frame 12 of the valve replacement device 10. For clarity, the valve element 14 has been omitted from the valve replacement device 10 in FIG. 4. It can be appreciated from FIG. 4 that the expandable stent frame 12 may include a proximal anchor region 22 and a distal valve region 24. As described herein, it can be appreciated that the a proximal region of the expandable frame 12 may be configured to anchor the valve replacement device 10 and the distal region of the expandable frame 12 may be configured to house the valve element 14.

FIG. 4 further illustrates that the expandable frame 12 may include a plurality of strut members spaced around the central longitudinal axis 50 (see FIG. 1) of the expandable framework 12 to form the proximal anchor region 22. Additionally, the proximal anchor region 22 may further include a plurality of distal crowns 26 and one or more connector crowns 28 positioned along a distal end of the proximal anchor region 22. Further, the proximal anchor region 22 may further include a plurality of proximal crowns 30 and one or more proximally-extending attachment crowns 32 positioned along a proximal end of the proximal anchor region 22. It can be appreciated from FIG. 4 that the strut members forming the apex of the one or more attachment crowns 30 may extend proximally beyond the apex of each of the proximal crowns 30.

FIG. 4 further illustrates that the distal end region 24 of the expandable frame 12 may include one or more commissure assemblies 34 spaced around the central longitudinal axis 50 (see FIG. 1) of the expandable frame 12. For example, FIG. 4 illustrates three commissure assemblies 34 spaced equidistance around the central longitudinal axis 50 of the expandable frame 12. It can be appreciated that in some examples, the expandable frame 12 may include fewer than three commissure assemblies 34 while in other examples the stent frame 12 may include more than three commissure assemblies 34. For example, the stent frame 12 may include 1, 2, 3, 4, 5, 6, or more commissure assemblies 34 spaced around the central longitudinal axis 50 (see FIG. 1) of the expandable frame 12.

FIG. 4 further illustrates that each of the commissure assemblies 34 may include a commissure post 36, a first connector strut 38 and a second connector strut 40. Additionally, FIG. 4 illustrates that the first connector strut 38 of the commissure assembly 34 may include a first end attached to the first commissure post 36 and a second end attached to the first connector crown 28 at a pivot point 42. Additionally, the FIG. 4 illustrates that the second connector strut 40 of the commissure assembly 34 may include a first end attached to the first commissure post 36 and a second end attached to the first connector crown 28 at the pivot point 42.

It can be appreciated that each of the commissure assemblies 34 may configured to stabilize and support the valve element 14 when positioned in a blood vessel (e.g. the aorta). As shown in FIGS. 1-3, the three valve leaflets 20a, 20b, 20c of the valve element 14 of the valve replacement device 10 may be attached to each of the commissure posts 36 of the commissure assemblies 34. In some examples, each of the valve leaflets 20a, 20b, 20c of the valve element 14 may be fixed to its respective commissure post 36 by wrapping around and suturing the valve leaflet to the commissure post.

In some examples, each of the valve leaflets 20a, 20b, 20c may be sized and shaped such as to be insertable or attachable into attachment means provided on its respective commissure posts 36 of the expandable frame 12. For example, each of the commissure posts 36 may include fixation means for the leaflets 20a, 20b, 20c represented by an aperture 44 and openings 46. The aperture 44 in the form of a longitudinal hole is positioned substantially in the center of the commissure post 36. The aperture 44 may be shaped and sized such as to allow insertion of at least a portion of one or more of the valve leaflets 20a, 20b, 20c. Additionally, the aperture 44 is flanked on both sides by a plurality of openings 46. An additional opening 46 is positioned on top of the aperture 44. The openings 46 may be utilized to thread suture wire therethrough when attaching a portion of the valve leaflets 20a, 20b, 20c to their respective commissure posts 36. In some examples (such as the example valve 10 shown in FIGS. 1-3), a portion of one or more of the valve leaflets 20a, 20b, 20c may be folded around and sutured to their respective commissure post 36, thereby covering each of the commissure posts 36.

FIG. 4 further illustrates that the inflow portion of the expandable frame 12 may include a zig-zag shape defined by cells of a lattice structure including at least one row of lattice cells. The zig-zag shape may be defined by alternating free apices 30, 32 and connected apexes 54. For example, FIG. 4 illustrates that the proximal anchor region 22 may be formed by a plurality of strut members which are arranged to form a lattice structure including a first row of cells 48 positioned distal to a second row of cells 52. Additionally, as discussed herein, one or more proximally-extending attachment crowns 32 may be interspersed with the proximal crowns 30. In other words, FIG. 4 illustrates that a stent strut apex forming an attachment crown 32 may be bordered on either side by stent struts which form an apex of a proximal crown 30, whereby apex forming the attachment crown 32 may extend proximally of the apices forming the proximal crowns 30. As discussed herein, the proximal anchor region 22 may serve to anchor the valve replacement device 10 in the native annulus, or towards the ventricular side of the valve replacement device 10.

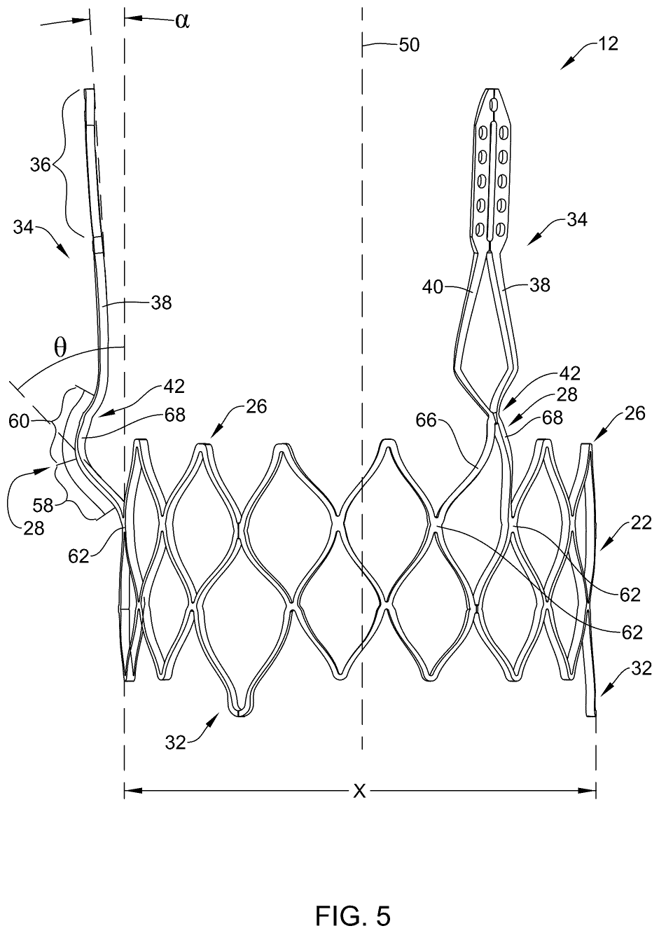

FIG. 5 illustrates a side view of a portion of the expandable frame 12. For clarity, the portion of the expandable frame 12 illustrates two of the three commissure post assemblies 34 described in FIG. 4. FIG. 5 further illustrates a side view of one commissure post assembly 34 including the commissure post 36 and the first connector strut 38 attached to the second connector strut 40 at the pivot point 42.

As discussed herein, FIG. 5 further illustrates the proximal anchor region 22 including the plurality of distal crowns 26 and one or more connector crowns 28 positioned along a distal end of the proximal anchor region 22. The connector crowns 28 may be positioned proximal to the pivot points 42. As discussed herein, the connector crown 28 may be positioned along a distal end of the proximal anchor region 22. FIG. 5 further illustrates that each connector crown 28 may be formed from a first connector crown strut 66 and a second connector crown strut 68. Further, the first connector crown strut 66 may include a first end attached to the first connector strut 38 and the second connector strut 40 at the pivot point 42. Similarly, the second connector crown strut 68 may include a first end attached to the first connector strut 38 and the second connector strut 40 at the pivot point 42. In other words, the first connector strut 38, the second connector strut 40, the first connector crown strut 66 and the second connector crown strut 68 may all be connected together at the pivot point 42.

FIG. 5 further illustrates that each of the first connector crown struts 66 and the second connector crown struts 68 may each include a second end attached at a distal attached apex 62 of the proximal anchor region 22. Additionally, FIG. 5 illustrates that each of the first connector crown struts 66 and the second connector crown struts 68 may include a first strut portion 58 and a second strut portion 60. It can be appreciated from the side view of the second connector crown strut 68 that the first strut portion 58 may be a substantially straight strut portion which forms an angle θ with the longitudinal axis 50 of the expandable frame 12. In other words, the first strut portion 58 of the first connector crown strut 66 and the second connector crown strut 68 may be angled away from the longitudinal axis 50, thereby extending the connector crown 28 radially outward of the diameter X of the plurality of struts defining the proximal anchor region 22. In some examples, the angle θ may be about 0 degrees to about 10 degrees, or about 5 degrees to about 15 degrees, or about 10 degrees to about 20 degrees, or about 15 degrees to about 25 degrees, or about 10 degrees to about 30 degrees, or about 15 degrees to about 35 degrees, or about 20 degrees to about 45 degrees, or about 25 degrees to about 45 degrees, or about 30 degrees to about 45 degrees, or about 35 degrees to about 55 degrees, or about 40 degrees to about 60 degrees, or about 45 degrees to about 55 degrees, or about 50 degrees.

Additionally, it can be appreciated from the side view of the connector crown 28 shown in FIG. 5 that the second strut portion 60 of the second connector crown strut 68 and the first connector crown strut 66 may be curved, thereby redirecting the second strut portion 60 of the second connector crown strut 68 and the first connector crown strut 66 to curve back toward the longitudinal axis 50 of the expandable frame 12. Accordingly, it can be appreciated that the inner surfaces of the first strut portion 58 and the second strut portion 60 (of each of the second connector crown strut 68 and the first connector crown strut 66) facing toward the longitudinal axis 50 define a convex surface relative to the longitudinal axis 50 of the expandable frame 12.

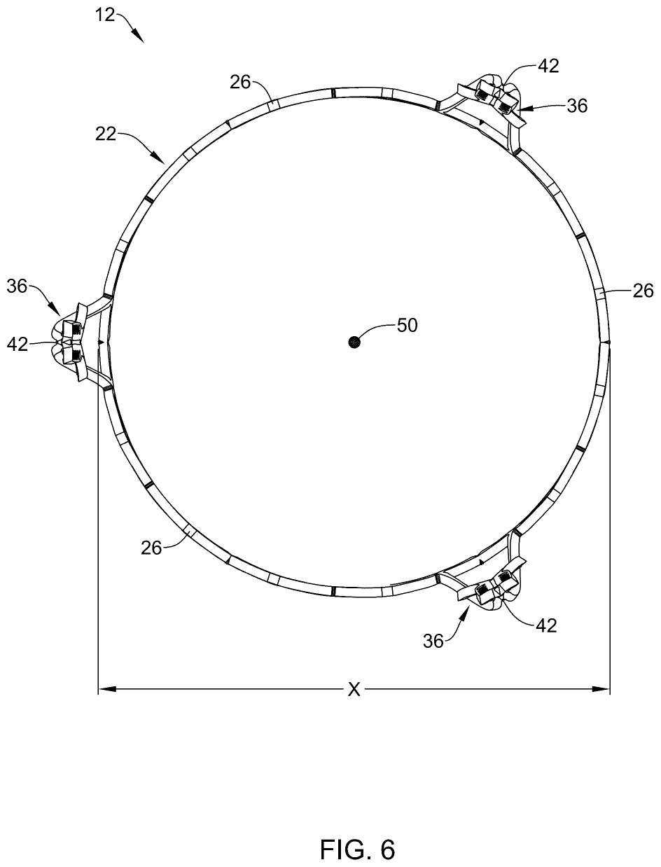

FIG. 5 further illustrates that the first connector strut 38, the second connector strut 40 and the commissure post 36 of each commissure assembly 34 together may be angled α degrees away from the longitudinal axis 50 of the expandable frame 12. In some examples, the angle α may be about 0 degrees to about 20 degrees, or about 1 degrees to about 15 degrees, or about 2 degrees to about 14 degrees, or about 3 degrees to about 13 degrees, or about 4 degrees to about 12 degrees, or about 5 degrees to about 11 degrees, or about 6 degrees to about 10 degrees, or about 8 degrees to about 12 degrees, or about 5 degrees to about 10 degrees, or about 8 degrees. In other words, as shown in a top view of the expandable frame 12, FIG. 6 illustrates that the first connector strut 38, the second connector strut 40 and the commissure post 36 of each commissure assembly 34 together may be angled away from the longitudinal axis 50 whereby the commissure post 36 of each commissure assembly 34 of the frame 12 extends radially outward of the diameter X defined by the distal crowns 26 of the proximal anchor region 22.

It can be appreciated that it may be desirable to design the valve replacement device 10 disclosed herein to be compliant such that the proximal anchor region 22 may substantially conform to and/or be in sealing engagement with the shape and/or geometry of the lateral walls of a native valve annulus within which the valve replacement device 10 is deployed. In some embodiments, the valve replacement device 10 may expand to a size, extent, or shape different from a maximum unconstrained extent, as determined by the surrounding tissue and/or lateral wall of the native valve annulus within which it is deployed. Further, it can be appreciated that elements of the expandable framework 12 may be tailored to increase the flexibility and compliance of the proximal anchor region 22 of the expandable framework 12 of the valve replacement device 10, thereby permitting the proximal anchor region 22 of the expandable framework 12 of the valve replacement device 10 to conform to the tissue around it, rather than forcing the tissue to conform to the expandable framework 12 and/or the valve replacement device 10. Additionally, it may be desirable to design the valve replacement device 10 discussed herein to include various features which improve the sealing capabilities of the proximal anchor region 22 of the valve replacement device 10 without compromising the Effective Orifice Area of the valve element 14 when positioned within a native valve annulus of a patient.

Accordingly, designing the expandable framework 12 of the valve replacement device 10 to maximize an effective orifice area of the valve element 16 while permitting the proximal anchor region 22 to conform to and/or be in sealing engagement with the shape and/or geometry of the lateral walls of a native valve annulus includes designing the expandable framework 12 such that there is a single mechanical link between each of the distal commissure assemblies 34 and the connector crown 28 to which each respective distal commissure assembly is attached. As discussed herein and illustrated in FIGS. 4-5, each pivot point 42 defines a single link between the distal commissure assemblies 34 and the connector crown 28 to which each respective distal commissure assembly is attached.

Further, designing the expandable framework 12 of the valve replacement device 10 to maximize an effective orifice area of the valve element 16 while permitting the proximal anchor region 22 to conform to and/or be in sealing engagement with the shape and/or geometry of the lateral walls of a native valve annulus may also include flaring each of the distal commissure assemblies 34 radially away from the longitudinal axis 50 of the expandable framework 12. As discussed herein and illustrated in FIGS. 4-5, each of the distal commissure assemblies may be angled about 0 degrees to about 20 degrees, or about 1 degrees to about 15 degrees, or about 2 degrees to about 14 degrees, or about 3 degrees to about 13 degrees, or about 4 degrees to about 12 degrees, or about 5 degrees to about 11 degrees, or about 6 degrees to about 10 degrees, or about 8 degrees to about 12 degrees, or about 5 degrees to about 10 degrees, or about 8 degrees away from the longitudinal axis 50 of the expandable framework 12.

Further, designing the expandable framework 12 of the valve replacement device 10 to maximize an effective orifice area of the valve element 16 while permitting the proximal anchor region 22 to conform to and/or be in sealing engagement with the shape and/or geometry of the lateral walls of a native valve annulus may also include extending a portion of the connector crowns 28 radially away from the longitudinal axis 50 of the expandable framework 12. As discussed herein and illustrated in FIGS. 4-5, the first strut portion 58 of the first connector crown strut 66 and the second connector crown strut 68 may be angled away from the longitudinal axis 50, thereby extending the connector crown 28 radially away from the longitudinal axis 50 of the expandable framework 12. Additionally, in some examples, the thickness of the struts forming the proximal anchor region 22 may be about 0.30 millimeters (mm) (0.0118 inches) to about 0.50 mm (0.0197 inches), or about 0.35 mm (0.0138 inches) to about 0.45 mm (0.0177 inches), or about 0.40 mm (0.0157 inches) thick. It can be appreciated that extending the connector crown 28 radially away from the longitudinal axis 50 of the expandable framework 12 and/or designing the struts of the proximal anchor region 22 to be about 0.40 mm (0.0157 inches) thick may permit the proximal anchor region 22 to conform to and/or be in sealing engagement with the shape and/or geometry of the lateral walls of a native valve annulus, thereby improving sealing and decreasing the likelihood of migration of the valve replacement device 10 into the ventricle.

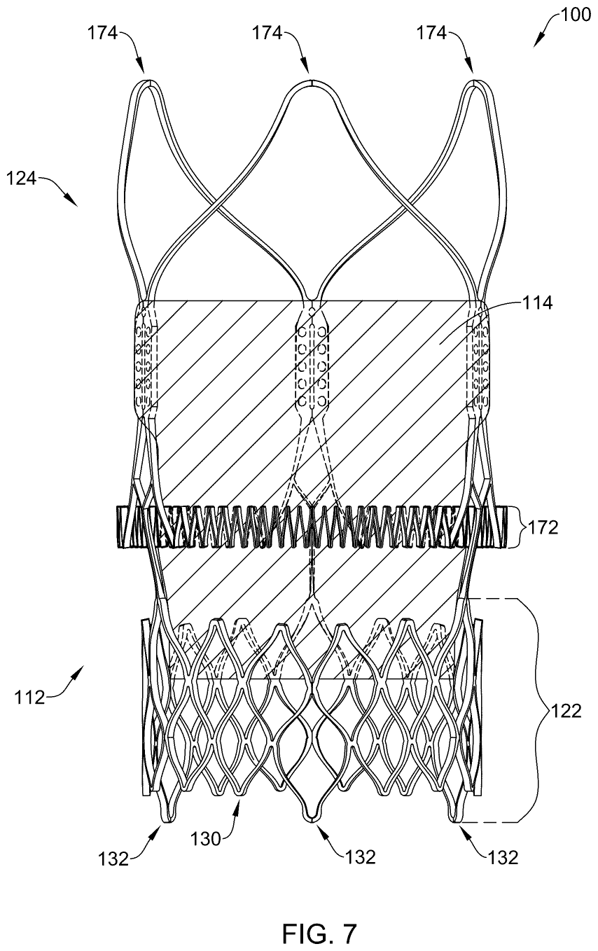

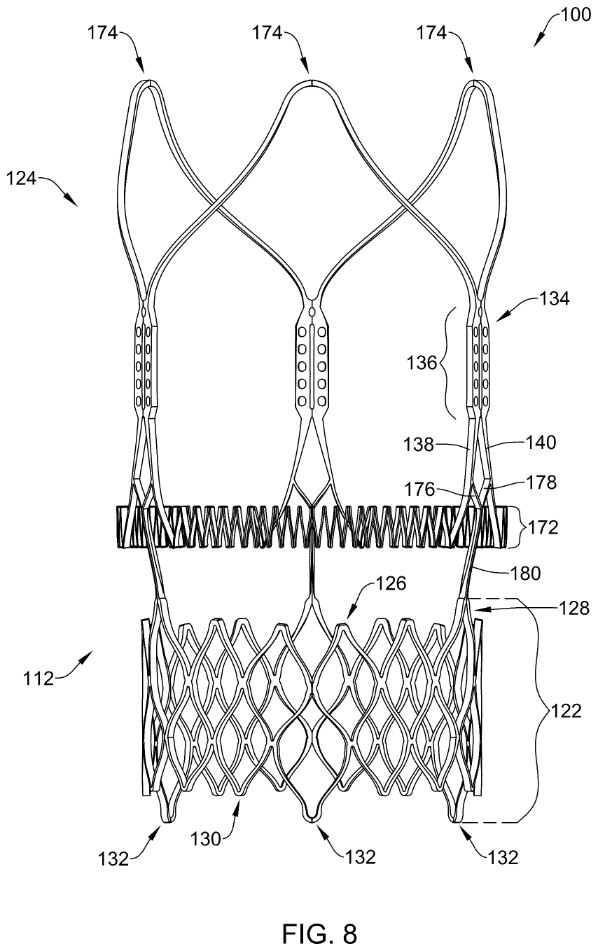

FIG. 7 illustrates another example valve replacement device 100 including an expandable frame 112 (e.g., a stent frame) and a valve element 114. The valve replacement device 100 may be similar in form and function to the valve replacement device 10 described herein. For example, the valve replacement device 100 may include a valve element positioned inside the frame 112, whereby the valve element 114 may be attached to one or more commissure posts 136 of a commissure assembly 134 (shown in FIG. 8). In some examples, one or more valve leaflets of the valve element 114 may be fixed to a commissure post 36 by wrapping around and suturing the valve leaflet to the commissure post 136. It can be appreciated that each of the commissure assemblies 134 may configured to stabilize and support the valve element 114 when positioned in a blood vessel (e.g. the aorta).

The valve replacement device 100 may be configured to be inserted by a transfemoral approach, but the device may also be inserted generally by another transvascular approach or by a transapical approach. The valve replacement device 100 may be collapsed within a delivery catheter for tracking and deploying the valve replacement device 100 within the heart and/or the aorta. The delivery catheter may be advanced over a guidewire to a valve treatment site within the patient's heart. The delivery catheter may include a distal sheath component for constraining the valve replacement device 100 in a compressed state for delivery, the sheath arrangement being operable to un-sheath the valve replacement device 100 to allow the expandable frame 112 to expand to its functional state and nest within the native annulus of the patient.

The terms “distal” and “proximal” are used herein to designate the parts of the valve replacement device 100 or of its components lying further away or closer to the heart, respectively. Accordingly, FIG. 7 illustrates that the valve replacement device 100 may include a distal end which may be intended to be positioned in an artery and a proximal end which may be intended to be positioned in or towards the ventricle of the heart of a patient. The distal end may also be referred to as the aortic end and the proximal end as the ventricular end. When the valve replacement device 100 is in place, blood may flow from the proximal end toward the distal end. Accordingly, the region of the valve replacement device 100 adjacent the proximal may be referred to as the “inflow section” and the region of the valve replacement device 100 adjacent the distal end may be referred to as the “outflow section.”

FIG. 7 further illustrates that the valve replacement device 100 may include a proximal region configured to anchor the valve replacement device 100 and a distal region configured to house a valve element 114. For a supra-annular valve replacement device 100, the primary function of the proximal region of the valve replacement device 100 may be to anchor the valve replacement device 100 and to create a seal against the native annulus/anatomy, while the function of the distal end of the valve replacement device 100 may be to support the valve leaflet function.

FIG. 7 further illustrates that the expandable frame 112 may include a plurality of stabilization arches 174. For example, FIG. 7 illustrates that the expandable frame 112 may include three stabilization arches 174 circumferentially spaced around the longitudinal axis of the expandable frame 112. However, this is not intended to be limiting. Rather, the expandable frame 112 may include 1, 2, 3, 4, 5, 6 or more stabilization arches 174. The stabilization arches 174 may stabilize the expandable frame 112 in a blood vessel, preferably the aorta, during deployment. The stabilization arches 174 may be attached with their proximal end directly to a distal end of a commissural posts 134 (shown in FIG. 8). Starting from the proximal end the stabilization arches 174 may diverge radially outwardly over a part of their length and converge radially inwardly towards their distal end.

FIG. 8 further illustrates that the expandable frame 112 may include a plurality of strut members forming a proximal anchor region 122. The proximal anchor region 122 may be similar in form and function to the proximal anchor region 22 described herein. The proximal anchor region 122 may include a plurality of distal crowns 126 and one or more connector crowns 128 positioned along a distal end of the proximal anchor region 122. Further, the proximal anchor region 122 may further include a plurality of proximal crowns 130 and one or more proximally-extending attachment crowns 132 positioned along a proximal end of the proximal anchor region 122. It can be appreciated from FIG. 8 that the strut members forming the apex of the one or more attachment crowns 130 may extend proximally beyond the apex of each of the proximal crowns 130.

FIG. 8 illustrates the expandable frame 112 of the valve replacement device 100. However, for clarity, the valve element 114 has been omitted from the valve replacement device 100 in FIG. 7. It can be appreciated from FIG. 8 that the expandable stent frame 112 may include a proximal anchor region 122 and a distal valve region 124. As described herein, it can be appreciated that the a proximal region of the expandable frame 112 may be configured to anchor the valve replacement device 100 and the distal region of the expandable frame 112 may be configured to house the valve element 114.

FIG. 8 further illustrates that the distal end region 124 of the expandable frame 112 may include one or more commissure assemblies 134 circumferentially spaced around the expandable frame 112. For example, FIG. 8 illustrates three commissure assemblies 134 spaced equidistance around the expandable frame 112. It can be appreciated that in some examples, the expandable frame 112 may include fewer than three commissure assemblies 134 while in other examples the stent frame 112 may include more than three commissure assemblies 134. For example, the stent frame 112 may include 1, 2, 3, 4, 5, 6, or more commissure assemblies 134 spaced around the expandable frame 112.

FIG. 8 further illustrates that each of the commissure assemblies 134 may include a commissure post 136, a first connector strut 138, a second connector strut 140, a third connector strut 176 and a fourth connector strut 178. Additionally, FIG. 8 illustrates that the first connector strut 138 of the commissure assembly 134 may include a first end attached to the commissure post 136 and a second end attached to a medial anchor region 172. Additionally, the FIG. 8 illustrates that the second connector strut 140 of the commissure assembly 134 may include a first end attached to the first commissure post 136 and a second end attached to a medial anchor region 172. The medical anchor region may include a plurality of zig-zag struts forming a series of apices and valleys circumferentially around the expandable stent frame 112. The medial anchor region 172 may include struts that extend radially outward to conform to and/or be in sealing engagement with the shape and/or geometry of the lateral walls of a native valve annulus, thereby improving sealing and decreasing the likelihood of migration of the valve replacement device 100 into the ventricle.

Additionally, FIG. 8 illustrates that a first end of the third connector strut 176 may be attached to the first connector strut 138 and a first end of the fourth connector strut 178 may be attached to the second connector strut 140. Further, FIG. 8 further illustrates a second end of the third connector strut 176 and a second end of the fourth connector strut 178 may be attached to a first end of a fifth connector strut 180. Additionally, FIG. 8 illustrates that a second end of the fifth connector strut 180 may be attached to the apex of a connector crown 128. Accordingly, the proximal anchor region 122 of the expandable frame member 112 may be attached to the distal valve region 124 via the first connector strut 138, the second connector strut 140, the third connector strut 176, the fourth connector strut 178 and the fifth connector strut 180.

The materials that can be used for the various components of the valve replacement device 10 and the various other valve replacement devices disclosed herein may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R 30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R 30003 such as ELGILOY®, PHYNOX®, and the like); platinum enriched stainless steel; titanium; combinations thereof; and the like; or any other suitable material.

In at least some embodiments, portions or all of the valve replacement device 10 and the various other valve replacement devices disclosed herein may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the valve replacement device 10 and the various other valve replacement devices disclosed herein in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the valve replacement device 10 and the various other valve replacement devices disclosed herein to achieve the same result.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A valve replacement device for transcatheter implantation, comprising:
an expandable framework including a plurality of strut members spaced around a central longitudinal axis of the expandable framework and forming a proximal anchor region, wherein the proximal anchor region includes a plurality of distal crowns and a first connector crown positioned along a distal end of the proximal anchor region;
a first commissure assembly coupled to the distal end of the proximal anchor region, wherein the first commissure assembly includes a first commissure post, a first connector strut and a second connector strut; and
one or more valve leaflets mounted at least partly within the expandable framework;
wherein the first connector strut includes a first end attached to the first commissure post and a second end attached to the first connector crown at a first pivot point;
wherein the second connector strut includes a first end attached to the first commissure post and a second end attached to the first connector crown at the first pivot point;
wherein the pivot point is positioned radially outward of the plurality of proximal crowns.

2. The valve replacement device of claim 1, wherein plurality of struts forming the proximal anchor region are configured to shift between a collapsed configuration and an expanded configuration.

3. The valve replacement device of claim 2, further comprising a second commissure assembly coupled to the distal end of the proximal anchor region, wherein the second commissure assembly includes a second commissure post, a second connector strut and a third connector strut.

4. The valve replacement device of claim 3, further comprising a third commissure assembly coupled to the distal end of the proximal anchor region, wherein the third commissure assembly includes a third commissure post, a fourth connector strut and a fifth connector strut.

5. The valve replacement device of claim 4, wherein the one or more valve leaflets include a first leaflet, a second leaflet and a third leaflet.

6. The valve replacement device of claim 5, wherein the first leaflet is coupled to the first commissure post and the second commissure post, wherein the second leaflet is coupled to the first commissure post and the third commissure post, and wherein the third leaflet is coupled to the second commissure post and the third commissure post.

7. The valve replacement device of claim 6, wherein the first leaflet, the second leaflet and the third leaflet are configured to shift from a sealed configuration to an open configuration.

8. The valve replacement device of claim 7, wherein the first leaflet, the second leaflet and the third leaflet define a maximum effective orifice area when in the open configuration.

9. The valve replacement device of claim 8, wherein the first commissure assembly, the second commissure assembly, and the third commissure assembly are configured to shift between a collapsed configuration and an expanded configuration.

10. The valve replacement device of claim 9, wherein the first commissure assembly, the second commissure assembly, and the third commissure assembly are configured to maintain the expanded configuration when the plurality of struts forming the proximal anchor region are in a partially collapsed configuration.

11. The valve replacement device of claim 10, wherein the first commissure assembly, the second commissure assembly, and the third commissure assembly are configured to maintain the maximum effective orifice when the plurality of struts forming the proximal anchor region are in a partially collapsed configuration.

12. The valve replacement device of claim 11, wherein the first commissure assembly, the second commissure assembly, and the third commissure assembly are configured to expand radially away from the central longitudinal axis when the struts forming the proximal anchor region are in the partially expanded configuration.

13. The valve replacement device of claim 12, wherein the first commissure assembly, the second commissure assembly, and the third commissure assembly are configured to extend away from the central longitudinal axis at an angle from 0 to 5 degrees.

14. The valve replacement device of claim 13, wherein the first commissure assembly pivots about the first pivot point to extend away from the central longitudinal axis at an angle from 0 to 5 degrees.

15. A valve replacement device for transcatheter implantation, comprising:
an expandable framework including a plurality of strut members spaced around a central longitudinal axis of the expandable framework and forming a proximal anchor region, wherein the proximal anchor region includes a plurality of distal crowns and a first connector crown positioned along a distal end of the proximal anchor region, and wherein the first connector crown includes a curved region which extends radially away from the central longitudinal axis;
a first commissure assembly coupled to the distal end of the proximal anchor region, wherein the first commissure assembly includes a first commissure post, a first connector strut and a second connector strut; and
one or more valve leaflets mounted at least partly within the expandable framework;
wherein the first connector strut includes a first end attached to the first commissure post and a second end attached to the first connector crown at a first pivot point;
wherein the second connector strut includes a first end attached to the first commissure post and a second end attached to the first connector crown at the first pivot point;
wherein the pivot point is positioned radially outward of the plurality of proximal crowns.

16. The valve replacement device of claim 15, further comprising a second commissure assembly and a third commissure assembly coupled to the distal end of the proximal anchor region, wherein the second commissure assembly includes a second commissure post, a second connector strut and a third connector strut, wherein the third commissure assembly includes a third commissure post, a fourth connector strut and a fifth connector strut.

17. The valve replacement device of claim 16, wherein the one or more valve leaflets include a first leaflet, a second leaflet and a third leaflet, and wherein the first leaflet is coupled to the first commissure post and the second commissure post, wherein the second leaflet is coupled to the first commissure post and the third commissure post, and wherein the third leaflet is coupled to the second commissure post and the third commissure post.

18. The valve replacement device of claim 17, wherein the first leaflet, the second leaflet and the third leaflet are configured to shift from a sealed configuration to an open configuration, and wherein the first leaflet, the second leaflet and the third leaflet define a maximum effective orifice area when in the open configuration.

19. The valve replacement device of claim 17, wherein the first commissure assembly, the second commissure assembly, and the third commissure assembly are configured to maintain the maximum effective orifice when the plurality of struts forming the proximal anchor region are in a partially collapsed configuration.

20. A valve replacement device for transcatheter implantation, comprising:
an expandable framework including a plurality of strut members spaced around a central longitudinal axis of the expandable framework and forming a proximal anchor region, wherein the proximal anchor region includes a plurality of distal crowns and a first connector crown positioned along a distal end of the proximal anchor region, and wherein the first connector crown includes a curved region which extends radially away from the central longitudinal axis;
a first commissure assembly coupled to the distal end of the proximal anchor region, wherein the first commissure assembly includes a first commissure post, a first connector strut and a second connector strut; and
one or more valve leaflets mounted at least partly within the expandable framework;
wherein the first connector strut includes a first end attached to the first commissure post and a second end attached to the first connector crown at a first pivot point;
wherein the second connector strut includes a first end attached to the first commissure post and a second end attached to the first connector crown at the first pivot point;
wherein the pivot point is positioned radially outward of the plurality of proximal crowns;
wherein the first commissure assembly is configured to pivot about the pivot point to extend radially away from the central longitudinal axis.
